(19) United States
(12) Patent Application Publication
(10) Pub. No.: US 2023/0417730 A1
(43) Pub. Date: Dec. 28, 2023

(54) METHOD FOR DETECTING AND/OR QUANTIFYING A METAL ELEMENT IN A BIOLOGICAL LIQUID

(71) Applicant: CARDIOVASCULAR LAB S.R.L., Milano (IT)

(72) Inventors: Vincenzo Mazzaracchio, Milano (IT); Fabiana Arduini, Milano (IT); Danila Moscone, Milano (IT); Francesco Chiara, Milano (IT); Simona Roggero, Milano (IT)

(21) Appl. No.: 18/252,896

(22) PCT Filed: Nov. 17, 2021
(86) PCT No.: PCT/IB2021/060659

§ 371 Date: May 15, 2023

(30) Foreign Application Priority Data
Nov. 17, 2020 (IT) 102020000027546

(51) Int. Cl. G01N 33/487; G01N 27/48; G01N 33/49; G01N 27/327

(52) U.S. Cl. CPC G01N 33/48707; G01N 27/48; G01N 33/491; G01N 27/327

(57) ABSTRACT

The present invention concerns a method for detecting and/or quantifying a metal element in a biological liquid, in particular selected from the group consisting of blood, plasma and serum, comprising the steps of: contacting the biological liquid with at least one fluorinated acid substance; applying the biological liquid and the fluorinated acid substance to an electroanalytical sensor; and detecting by means of the electroanalytical sensor a current signal proportional to the amount of metal element in the biological liquid.

### CROSS-REFERENCE TO RELATED APPLICATIONS

This patent application claims priority from Italian patent application no. 102020000027546 filed on 17 Nov. 2020, the entire disclosure of which is incorporated herein by reference.

### TECHNICAL FIELD

The present invention concerns a method for detecting and/or quantifying a metal element in a biological liquid (whole blood, serum, plasma, urine, saliva, sweat, breast milk), preferably selected from the group consisting of blood, plasma and serum, comprising contacting the biological liquid with at least one fluorinated acid substance and detecting by means of an electroanalytical sensor a current signal proportional to the amount of metal element in the biological liquid.

### STATE OF THE ART

The possibility of detecting and quantifying metal ions in biological liquids such as, for example, blood, plasma and serum, in a simple and inexpensive manner is of fundamental importance in diagnostics both in the context of systems for “out of the lab” diagnosis (point-of-care devices for home care, or in pharmacies and surgeries), and in diagnostic laboratories.

Sensor technologies with electrochemical detection modes designed specifically for measuring metals, in particular iron, in the blood are currently not widely available on the market. Due to the physical-chemical characteristics of iron, however, an electrochemical method would guarantee a higher accuracy with respect to the colorimetric methods currently in use. For the determination of iron, in fact, the gold standard used in the laboratory is by atomic absorption or, more frequently, with the colorimetric method that uses the Ferene technique (most widespread). The method with Ferene-based colorimetric technique was described for the first time in 1984 (Serum Iron Determination Using Ferene Triazine—Frank E. Smith and John Herbert). This method uses at least two reagents and an ionophore substance (Ferene) binding the iron to perform the colorimetric measurement. It is therefore a complex method that requires time and costly machines, with low-specificity results and poor sensitivity.

There is therefore the need to develop a method that allows detection and quantification of the metal elements in biological liquids that can be applied both outside the laboratory and in the laboratory in emergency situations or in the case of small poorly equipped facilities. In particular, there is a strong demand for a method that is less costly, quicker and simpler, specific and selective and adaptable to non-specialized laboratories and environments in diagnostic terms. This demand is particularly felt in the case of the metal element iron.

Electrochemical sensors have been developed which can be made on polyester or cellulose supports, in particular paper, that represent both an inexpensive and environment-friendly solution. However, these sensors still require optimization, in particular for use in complex matrices like blood.

### DISCLOSURE OF INVENTION

One object of the present invention is therefore to provide a method for detecting and/or quantifying a metal element, in particular iron, in a biological liquid preferably selected from the group consisting of blood, plasma and serum, which allows the above-mentioned problems to be solved simply and efficiently.

This object is achieved by means of the present invention relative to a method as defined in claim 1.

A further object of the present invention is to provide the use of a fluorinated acid substance, in particular trifluoroacetic acid (TFA), to detect and/or quantify a metal element in a biological liquid by means of an electroanalytical sensor as defined in claim 12.

### BRIEF DESCRIPTION OF THE FIGURES

FIG. 1 shows a schematic view of an example of an electroanalytical sensor used in the present invention.

FIG. 2 shows an example image of the process of modification according to a preferred embodiment of the working electrode of the electroanalytical sensor illustrated in FIG. 1.

FIG. 3 shows the phases of the method for production of the electroanalytical sensor of FIG. 1.

FIG. 4 illustrates a schematic image of the measurement system for measuring the electrochemical signal by means of the electroanalytical sensor of FIG. 1.

FIG. 5A illustrates a potential-current graph for different known quantities of Fe³⁺ and FIG. 5B the relative calibration curve for Fe³⁺ using the electroanalytical sensor of FIG. 1.

FIG. 6 illustrates a graph with the calibration curve for Fe²⁺ using the electroanalytical sensor of FIG. 1.

FIG. 7 illustrates a schematic image of the method of detecting and quantifying iron in serum by means of the electroanalytical sensor of FIG. 1.

FIG. 8 illustrates a graph with the quantification curves of the iron in serum by means of the electroanalytical sensor of FIG. 1 in the presence of a fluorinated acid substance (trifluoroacetic acid, TFA) and in the presence of a non-fluorinated substance (trichloroacetic acid, TCA).

FIG. 9 illustrates a graph with the quantification curve of the copper in serum by means of the electroanalytical sensor of FIG. 1 in the presence of a fluorinated acid substance (trifluoroacetic acid, TFA) and with the working electrode modified with gold nanoparticles.

FIG. 10A and FIG. 10B illustrate a graph with the quantification curves of iron in serum (FIG. 10A) and in whole blood (FIG. 10B) respectively by means of the electroanalytical sensor of FIG. 1 in the presence of a fluorinated acid substance (trifluoroacetic acid, TFA).

FIG. 11 illustrates a potential-current graph for two known quantities of Fe²⁺ using an electroanalytical sensor analogous to that of FIG. 1 but with polyester instead of paper support.

FIG. 12 illustrates a graph with the quantification curves of iron in serum by means of the electroanalytical sensor of FIG. 1 in the presence of a fluorinated acid substance (trifluoropropionic acid).

FIG. 13 illustrates a graph with the quantification curves of iron (Fe²⁺ and Fe³⁺) in serum by means of the electroanalytical sensor of FIG. 1 in the presence of sulfonated trifluorostyrene.

FIG. 14 illustrates a graph with the quantification curves of iron in serum by means of the electroanalytical sensor of FIG. 1 without separation passages for separating the protein-containing fraction of the serum.

### DETAILED DISCLOSURE OF THE INVENTION

The method for detecting and/or quantifying a metal element in a biological liquid, preferably selected from the group consisting of blood, plasma and serum, according to the present invention comprises the following steps: contacting the biological liquid with at least a fluorinated acid substance, applying the biological liquid and the fluorinated acid substance to an electroanalytical sensor or to a polarograph; detecting by means of the electroanalytical sensor or polarograph a current signal proportional to the quantity of metal element in the biological liquid.

Preferably, the method according to the present invention comprises the following steps: —contacting the biological liquid with at least a fluorinated acid substance; separating a protein-containing fraction of the biological liquid from a fraction of the biological liquid comprising the metal element and the at least one fluorinated acid substance; applying the fraction of the biological liquid comprising the metal element and the fluorinated acid substance to an electroanalytical sensor or to a polarograph; detecting by means of the electroanalytical sensor or polarograph a current signal proportional to the quantity of metal element in the biological liquid.

The method works with any type of electrochemical detection. In addition to the technique used in the examples (square wave voltammetry) it is possible to use other electrochemical techniques, specifically linear sweep voltammetry (LSV), normal pulse voltammetry (NPV) or differential pulse voltammetry. In the first case the potential applied varies linearly over time, increasing in a linear proportional manner over time. In the second case, pulses are applied with amplitude gradually increasing over time. Lastly, in the DPV, to generate the potential signal, a series of fixed amplitude pulses are used, along a linear scale. Lastly, stripping techniques can be used, namely techniques in which a fixed reduction (or oxidation) potential is firstly applied to pre-concentrate and deposit the metal in question on the surface of the working electrode. Subsequently a potential is applied in the form of one of the previously described voltammetry techniques for detecting the metal. The electroanalytical method can also be applied to polarographic systems that use adsorption working electrodes (graphite).

The metal element is preferably selected from the group consisting of iron, copper, selenium, zinc, manganese, caesium, rubidium, lead, cadmium and mercury. More preferably, the metal element is iron or copper. Even more preferably the metal element is iron. In particular, the method according to the invention allows detection and quantification of both the Fe²⁺ and the Fe³⁺.

The at least one fluorinated acid substance can be trifluoroacetic acid (TFA), trifluoropropionic acid, monofluoroacetic acid (MFA) or difluoroacetic acid (DFA). Preferably it is trifluoroacetic acid (TFA).

According to the present invention it has been shown for the first time that trifluoroacetic acid (TFA), like the trifluoropropionic acid, monofluoroacetic acid (MFA) and difluoroacetic acid (DFA), can be used effectively to detect and/or quantify a metal element in a biological liquid selected from the group consisting of blood, plasma and serum by means of an electroanalytical sensor.

When TFA is used, the concentration of TFA in the biological liquid preferably ranges from 240 to 280 millimoles, more preferably 260 millimoles.

In addition to trifluoroacetic acid (TFA), a fluorinated polymer, preferably a fluoropolymer-copolymer consisting of sulfonated tetrafluoroethylene, sulfonated perfluorovinylether or sulfonated trifluorostyrene is preferably also used. In particular the polymer commercially known as Nafion (CAS number: 31175-20-9) can be used. Nafion is preferably used in the production of a preferred form of the sensor, directly on the working electrode as indicated in FIG. 2; alternatively or additionally, it can also be added directly to the sample to be examined. In the latter embodiment, Nafion is used preferably in a molar ratio from 0.1 to 10 with respect to TFA. The use of TFA in association with Nafion may possibly contribute to widening some properties of TFA. The acid treatment (together if necessary with centrifugation which will be discussed below) eliminates the protein-containing part of the biological liquid and allows optimal denaturation, also increasing the sensitivity of the method. TFA may also form complexes with the Nafion, which allow increase of the conduction generated by the analyte reduction current at the electrode/solution interface.

In a preferred embodiment, the step of separating the protein-containing fraction of the biological liquid from the fraction of the biological liquid comprising the metal element and the at least one fluorinated acid substance is carried out by means of centrifugation or ultracentrifugation, preferably ultracentrifugation. This embodiment is particularly suited to laboratory use.

In a preferred alternative embodiment, the step of separating the protein-containing fraction of the biological liquid from the fraction of the biological liquid comprising the metal element and the at least one fluorinated acid substance is carried out by means of a microfluidic system. In this embodiment also Nafion is preferably used, directly added to the sample to be analysed. In particular Nafion is added to the first fluorinated acid substance in the step of contacting the biological liquid with at least one fluorinated acid substance. Alternatively to a microfluidic system, membranes, beads and/or filters integrated in the surface of the electroanalytical sensor can be used. This embodiment is particularly suitable for use at the point-of-care, out-of-the-lab.

Although different types of electroanalytical sensors can be used, a preferred sensor is a sensor that comprises a polyester support and another preferred sensor is a sensor that comprises a support made of cellulose or derivatives thereof, on which a hydrophobic area delimits a hydrophilic working area, said hydrophilic working area comprising at least a working electrode, a reference electrode and a counter-electrode printed by screen-printing. The sensors can be obtained also by means of other methods such as, for example, inkjet printing, photolithography, chemical vapour deposition and electron-beam evaporation.

This type of sensor printed on cellulose has been described in the Italian patent application no. 102020000002017. Unlike the sensor described in the above-mentioned patent application, the sensor used in the present invention does not entail functionalization of the support with metal nanoparticles.

Preferably the support made of cellulose or derivatives thereof is formed of paper, in particular filter paper, Whatman paper or office paper, more preferably office paper. The hydrophobic area is preferably formed of wax printed on the support.

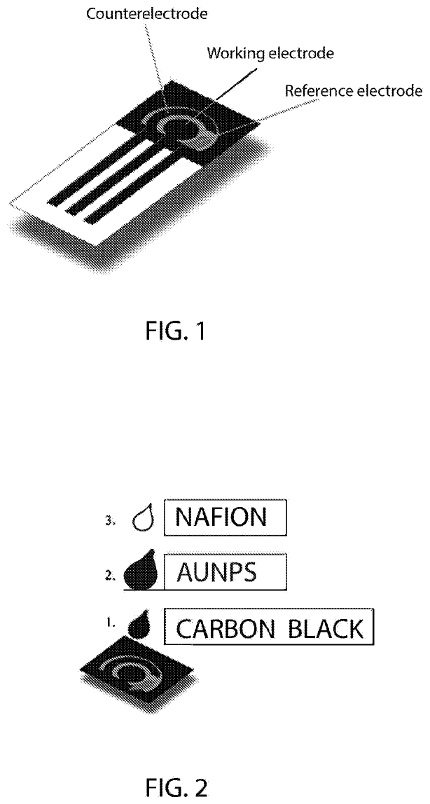

Preferably, the sensor has a configuration as illustrated in FIG. 1 with the circular-shaped working electrode having a surface area between 6 and 13 mm². However, it could have different shapes, for example square or rectangular, with dimensions up to 1 or 2 mm per side. The same electrode described in the invention can have a smaller diameter, reaching 1 mm in diameter.

Carbon black is preferably deposited on the working electrode. More preferably, metal nanoparticles of gold, palladium or platinum are deposited on the carbon black. Gold nanoparticles (AUNP) have proved to be particularly effective. Even more preferably a fluoropolymer-copolymer formed of sulfonated tetrafluoroethylene (for example, Nafion) is furthermore deposited on the carbon black and on any metal nanoparticles.

The preferred order of deposition on the working electrode is carbon black, metal nanoparticles and fluoropolymer-copolymer, as illustrated in FIG. 2.

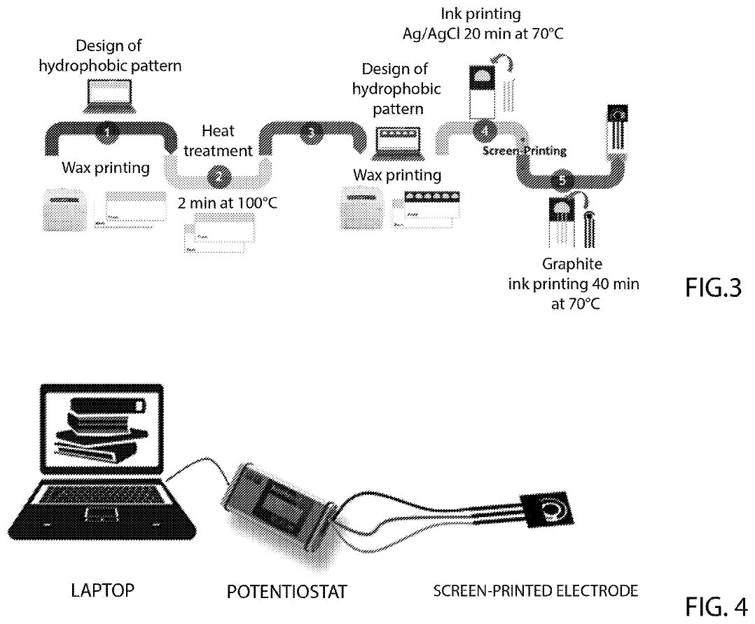

The preferred method for producing the sensor illustrated in FIG. 1 is the following. To print the electrodes the screen-printing technique is used, employing conductive inks based on graphite (working electrode and counterelectrode) and silver/silver chloride (reference electrode). The electrochemical cell is printed on paper made hydrophobic (colour blue) created by solid ink wax printer. The same cell is surrounded by a black outer hydrophobic part (again produced by solid ink wax printer). To produce the first blue hydrophobic area, the wax is treated at 100° C. so that it can permeate inside the paper. The process is illustrated in FIG. 3.

Alternatively to detecting by means of the electroanalytical sensor a current signal proportional to the amount of metal element in the biological liquid, the current signal can generate a change of colour in a chromophore and the detection can be colorimetric. In other words, by using the same analytical procedures described and the same electrochemical sensor, the current generated in the measurement is exploited to cause a chromophore to change colour. In this case the above-mentioned substance must be added as a final passage of the method and the final detection will be carried out by means of optical system. In the specific case of the detection of metals, substances that can be used are derivatives of N-ethylmaleimide.

### Example 1—Calibration of the Electroanalytical Sensor in Standard Solution

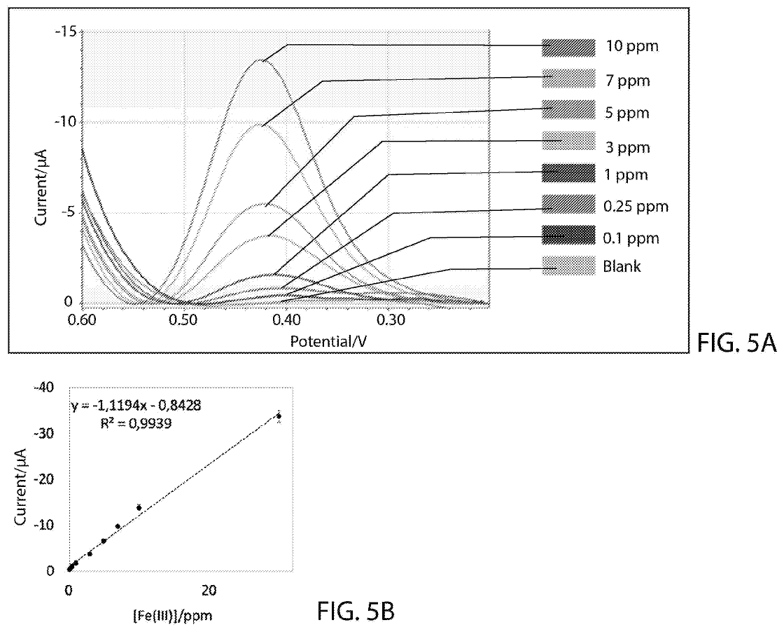

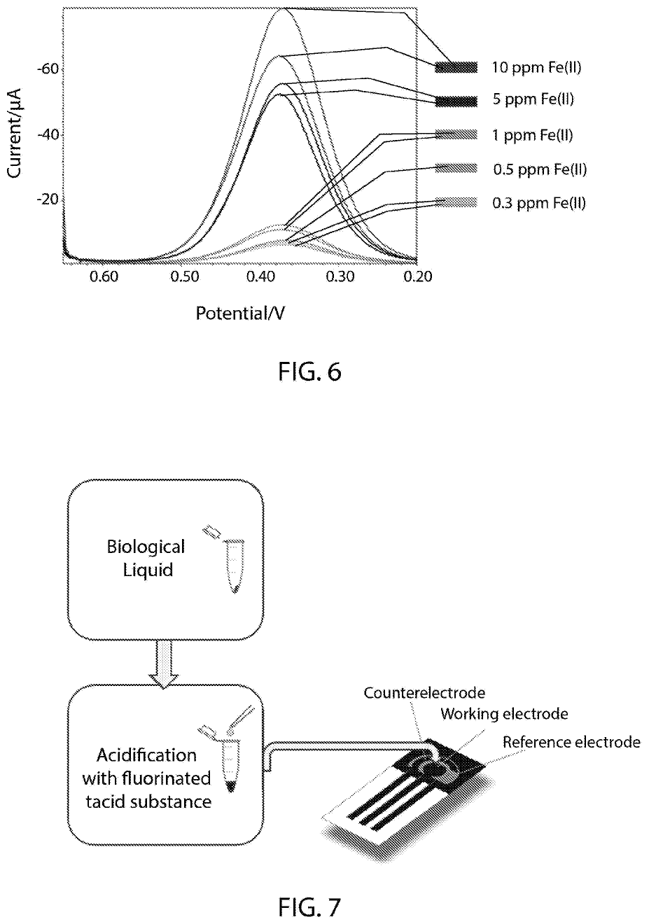

With reference to FIG. 4, 100 μl of solution containing known quantities of analyte were deposited on a sensor as described above and the current generated by reduction of the analyte was measured by means of potentiostat. The two calibration curves illustrated in FIGS. 5A and 5B and in FIG. 6 were then generated, for Fe³⁺ and Fe²⁺ respectively.

### Example 2

FIG. 7 illustrates the procedure according to a preferred embodiment in which the step of separating the protein-containing fraction of the biological liquid from the fraction of the biological liquid comprising the metal element and the at least one fluorinated acid substance is carried out by centrifugation or ultracentrifugation, preferably ultracentrifugation. The metal element detected and quantified is iron and the biological liquid is serum. The fluorinated acid substance used is TFA.

### Example 3

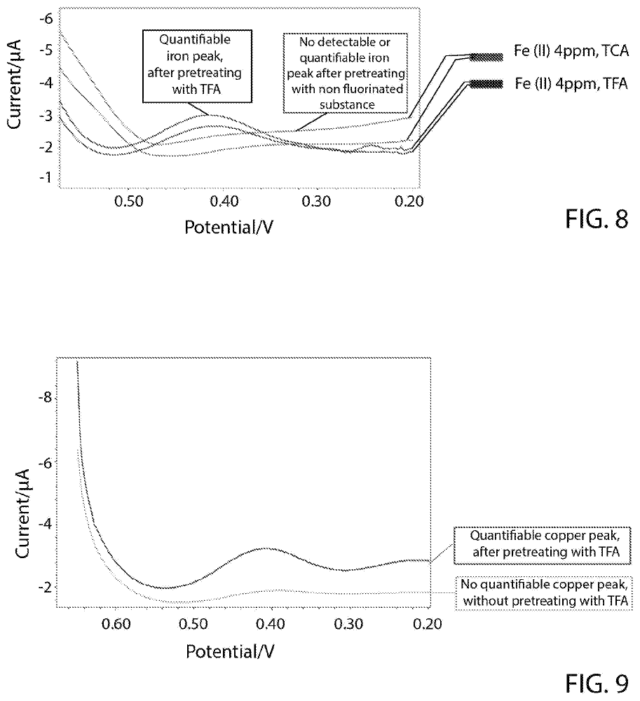

FIG. 8 illustrates a graph with the quantification curves of the iron in serum by means of the electroanalytical sensor of FIG. 1 in the presence of fluorinated acid substance (trifluoroacetic acid, TFA) and in the presence of a non-fluorinated substance (trichloroacetic acid, TCA). It is therefore evident that the method functions only in the presence of fluorinated acids and not in the presence of non-fluorinated acids.

### Example 4

FIG. 9 illustrates the results of an experiment of quantification of the copper in serum by means of the electroanalytical sensor described above in the presence of fluorinated acid substance (trifluoroacetic acid, TFA) and with the working electrode modified with gold nanoparticles.

### Example 5

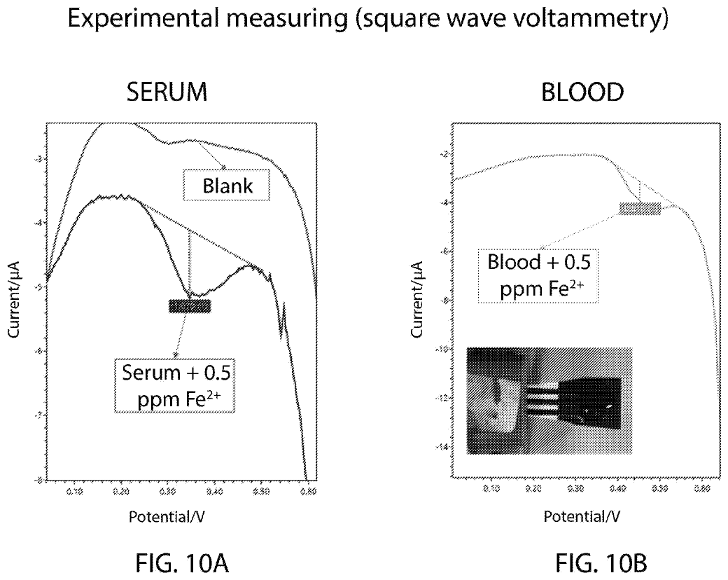

A test was performed to verify the iron measurement capacity according to the method of the present invention, also when the matrix is represented by whole blood instead of serum. FIG. 10A indicates measurement of the iron in the serum as already described; FIG. 10B indicates that the iron has been detected also in the whole blood. The procedure for demonstrating the capacity of the whole blood measurement system was carried out via the following steps: 1) addition of a quantity of iron at known concentration (0.5 ppm) to the whole blood (500 ml of whole blood), 2) addition of a quantity (10 uL) of TFA to the sample of whole blood, 3) centrifugation (12000 rpm for 10 min), 4) withdrawal of 100 uL of the supernatant, 5) deposit of the supernatant on the electrode surface to carry out the electrochemical measurement.

It was therefore shown that by using the same procedure it is possible to accurately measure the iron from whole blood. The rationale is that the iron bound to the haemoglobin has a very low concentration and does not “alter” determination of the serum iron, and is therefore potentially compatible with measurement at the point-of-care.

### Example 6

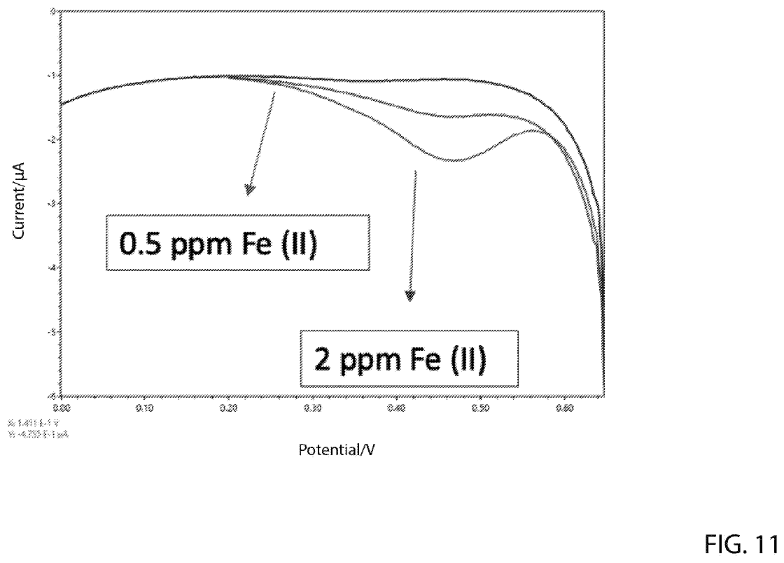

A test was performed to verify the iron measurement capacity by means of electrochemical sensor, also when the sensor is printed on polyester instead of paper. The procedure for demonstrating the capacity of the system to measure iron is the same as described. The sensor was tested by measuring standard solutions in the absence (upper line) and in the presence of 5 ppm (central line) and 2 ppm (lower line) of iron. The results are illustrated in FIG. 11.

### Example 7

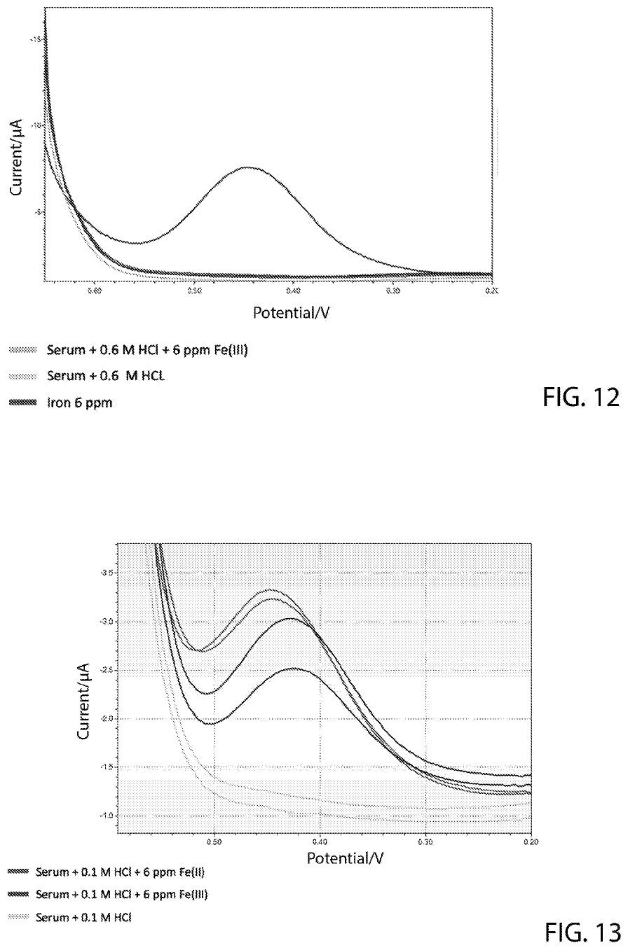

The method according to the invention was tested with trifluoropropionic acid, as illustrated in FIG. 12. The upper line indicates detection of the iron by means of electrochemical measurement. In the absence of trifluoropropionic acid, the iron present in the sample is not detected.

Analogous results (not illustrated for the sake of brevity) were obtained with monofluoroacetic acid and difluoroacetic acid.

### Example 8

The method according to the invention was tested with sulfonated trifluorostyrene, as illustrated in FIG. 13. Analogous results (not illustrated for the sake of brevity) were obtained with sulfonated perfluorovinylether.

### Example 9

The method according to the invention is particularly advantageous since it does not necessarily involve a phase of separation of the protein-containing fraction of the biological liquid. As shown in this example, the iron can be directly measured on whole blood.

The method entails the following steps:
1. Obtaining serum from the whole blood sample by means of centrifugation.
2. Addition of TFA to 500 μL of serum.
3. Deposit of the supernatant on the sensor. From the simple addition of TFA a precipitate forms, namely the supernatant.
4. Measurement by means of voltammetry and detection of the serum iron.

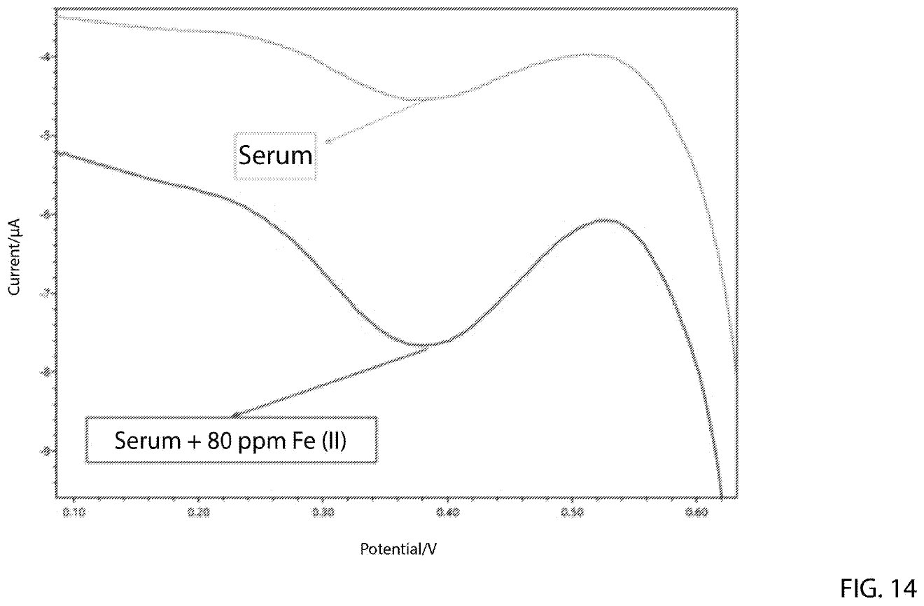

The test was carried out by measuring the serum iron on a serum sample as is (upper line) and on serum following the addition of iron 80 ppm (lower line). The results are illustrated in the graph of FIG. 14.

Advantages

With respect to the methods according to the prior art, the method according to the present invention has the following advantages:
lower cost (smaller investment for purchasing the necessary instruments and performing the analysis);
shorter execution time;
less need for personnel training;
less risk of exposure to toxic chemicals;
lower environmental impact;
improved specificity and selectivity of the method;
versatility (possibility of measuring different metal ions, possibility of detecting the electrochemical signal by detection of the change in current signal or by optical signal);
adaptability to both lab and surgery point-of-care methods.

1. A method for detecting and/or quantifying a metal element in a biological liquid, comprising the steps of:
contacting the biological liquid with at least one fluorinated acid substance;
applying the biological liquid and the fluorinated acid substance to an electroanalytical sensor or to a polarograph; and
detecting a current signal which is proportional to the amount of metal element in the biological liquid by means of the electroanalytical sensor or polarograph.

2. A method for detecting and/or quantifying a metal element in a biological liquid, comprising the steps of:
contacting the biological liquid with at least one fluorinated acid substance;
separating a protein-containing fraction of the biological liquid from a fraction of the biological liquid comprising the metal element and the at least one fluorinated acid substance;
applying the fraction of the biological liquid comprising the metal element and the fluorinated acid substance to an electroanalytical sensor or to a polarograph; and
detecting a current signal which is proportional to the amount of metal element in the biological liquid by means of the electroanalytical sensor or polarograph.

3. The method according to claim 1, wherein the electroanalytical sensor comprises a support made of cellulose, polyester or a derivative thereof, on which a hydrophobic area delimits a hydrophilic working area, said hydrophilic working area comprising at least one working electrode, one reference electrode and one counterelectrode printed by screen-printing.

4. The method according to claim 1, wherein the biological liquid is selected from the group consisting of blood, plasma, and serum.

5. The method according to claim 1, wherein the at least one fluorinated acid substance is trifluoroacetic acid (TFA).

6. The method according to claim 5, wherein a fluorinated polymer is used in addition to TFA.

7. The method according to claim 3, wherein the working electrode is treated with metal nanoparticles and/or a fluorinated polymer.

8. The method according to claim 2, wherein the step of separating the protein-containing fraction of the biological liquid from the fraction of the biological liquid comprising the metal element and the at least one fluorinated acid substance is carried out by centrifugation or ultracentrifugation.

9. The method according to claim 2, wherein the step of separating the protein-containing fraction of the biological liquid from the fraction of the biological liquid comprising the metal element and the at least one fluorinated acid substance is carried out by a microfluidic system.

10. The method according to claim 1, wherein the metal element is selected from the group consisting of iron, copper, selenium, zinc, manganese, caesium, rubidium, lead, cadmium, and mercury.

11. The method according to claim 10, wherein the metal element is Fe²⁺ and/or Fe³⁺.

12. A use of a fluorinated acid substance for detecting and/or quantifying a metal element in a biological liquid by means of an electroanalytical sensor.

13. The use according to claim 12, wherein the fluorinated acid substance is selected from the group consisting of trifluoroacetic acid, trifluoropropionic acid, monofluoroacetic acid (MFA) and difluoroacetic acid (DFA).

14. The use according to claim 13, wherein the fluorinated acid substance is trifluoroacetic acid.
